(19) United States
(12) Patent Application Publication
(10) Pub. No.: US 2021/0260349 A1
(43) Pub. Date: Aug. 26, 2021

(54) BALLOON-ASSISTED INFUSION TECHNIQUES

(71) Applicant: Transit Scientific, LLC, Salt Lake City, UT (US)

(72) Inventors: Shawn P. Fojtik, Park City, UT (US); Greg Method, San Francisco, CA (US)

(21) Appl. No.: 17/184,523

(22) Filed: Feb. 24, 2021

Related U.S. Application Data
(60) Provisional application No. 62/980,992, filed on Feb. 24, 2020.

(51) Int. Cl. A61M 25/10; A61B 17/3207

(52) U.S. Cl. CPC A61M 25/10184; A61B 17/320725; A61M 2025/109; A61M 2025/105; A61M 2025/1086; A61M 25/104

(57) ABSTRACT

A system for infusing fluid into a body of a subject includes an expandable medical device and a balloon catheter. The expandable medical device includes an expandable section that may be expanded and contracted and, when expanded, score a wall of a vessel or another open space within the body of the subject. The balloon catheter includes a balloon that expands when pressurized and can expand the expandable section. When pressure within the balloon is reduced, the expandable section can resiliently contract the balloon. The balloon may be shorter than the expandable section, enabling the balloon to be positioned at a plurality of locations along the length of the expandable section and to expand the expandable section to a plurality of different configurations. The system may be used to limit the flow of infused fluid beyond a target location within the body and/or to force fluid into the target location.

### CROSS-REFERENCE TO RELATED APPLICATION

A claim for the benefit of priority to the Feb. 24, 2020 filing date of U.S. Provisional Patent Application No. 62/980,992, titled BALLOON-ASSISTED INFUSION TECHNIQUES (“the '992 Provisional Application”) is hereby made pursuant to 35 U.S.C. § 119(e). The entire disclosure of the '992 Provisional Application is hereby incorporated herein.

### TECHNICAL FIELD

This disclosure relates generally to systems that include balloon catheters and medical devices that can be positioned over balloons to expand and contract with the balloons. This disclosure also relates to techniques for using to systems that include balloon catheters and expandable, contractible medical devices to deliver infuse drugs and other substances into a subject's body.

### SUMMARY

A system according to this disclosure may include a balloon catheter and an expandable medical device. The balloon catheter may comprise a percutaneous transluminal angioplasty (PTA) balloon catheter or the like. The expandable medical device may include an expandable section that fits over a balloon of the balloon catheter, expands and contracts with the balloon, and from which the balloon may be removed.

In some embodiments, a length of the expandable section of the expandable medical device may exceed a length of the balloon of the balloon catheter over which the expandable medical device is positioned. Such an arrangement may enable the balloon to slide back and forth within the expandable section, even when the balloon and, thus, the expandable section are in expanded states.

In a method of using a system according to this disclosure, the balloon catheter and the expandable medical device may be introduced into a body of a subject, with the expandable section of the expandable medical device and the balloon of the balloon catheter being advanced to a desired, or target, location within the subject's body. The balloon of the balloon catheter may then be expanded, expanding the expandable section of the expandable medical device. With the balloon and expandable section in an expanded state, fluid may be introduced into the vessel or other space, with flow of the fluid being prevented or limited by the balloon. The fluid may be introduced between the expandable medical device and the balloon catheter and exit the expandable medical device at a proximal end of the expandable section.

In some embodiments, the expanded balloon and expandable medical device may seal against a wall of the vessel (e.g., a blood vessel, etc.) or other space (e.g., an interior of a hollow organ, etc.) within which they are placed, preventing the fluid from moving distally beyond the balloon and expandable section. Alternatively, the balloon and expandable section may be inflated in a manner (e.g., by controlling pressure within the balloon) that enables fluid to flow distally beyond the balloon and expandable section, but controls the extent to which fluid flows distally beyond the balloon and expandable section (e.g., the rate at which fluid flows distally beyond the balloon and expandable section, the amount of fluid that flows distally beyond the balloon and expandable section, etc.).

In some embodiments, the balloon may be slightly deflated and re-inflated once or repeatedly to enable fluid to flow between the expandable section and the adjacent wall(s) of the vessel or other space within which they are placed, and to encourage the fluid to enter the wall(s) (e.g. by physically pushing the fluid into the wall, by repeatedly scoring the wall(s) with the expandable section, etc.).

Once infusion is complete, the balloon and expandable section may be contracted and withdrawn from the body of the subject.

In embodiments where the length of the expandable section of the expandable medical device exceeds a length of the balloon of the balloon catheter, the balloon may be moved back and forth within the expandable section. Such movement may occur as the balloon catheter is pushed distally and pulled proximally relative to the expandable section of the expandable medical device. For example, the balloon may be moved from a distal position within the expandable section to an intermediate position, then to a proximal position, then back to an intermediate position, and then back to a distal position. Alternatively, such movement may be from a proximal position, to an intermediate position, to a distal position, back to the intermediate position, and back to the proximal position. As another alternative, such movement may be peristaltic in nature, involving a plurality of different intermediate positions as the balloon is moved distally and or proximally. In yet another alternative, such movement may simply alternate between a proximal position and a distal position. Movement of the balloon may include decreasing a pressure within (i.e., partially deflating) the balloon, changing a location of the balloon within the vessel or other space and/or within the expandable section of the expandable medical device, and increasing a pressure within (i.e., further inflating) the balloon. Movement of the balloon within the expandable section may force fluid (e.g. a drug, another substance, etc.) toward and/or into or otherwise encourage the fluid to flow into a wall of the vessel or other space within which the balloon and expandable section are located.

Other aspects of the disclosure, as well as features and advantages of various aspects of the disclosed subject matter should be apparent to those of ordinary skill in the art through consideration of the foregoing disclosure.

### BRIEF DESCRIPTION OF THE DRAWINGS

In the drawings:

FIG. 1 illustrates an embodiment of a system, including a balloon catheter and an expandable medical device, within a vessel, with a balloon of the balloon catheter located in an expandable section of the expandable medical device and the balloon and expandable sections in unexpanded, or contracted, states;

FIG. 2 illustrates the embodiment of the system of FIG. 1 with the balloon of the balloon catheter and the expandable section of the expandable medical device in expanded states and the expandable section forced against a wall of the vessel;

FIG. 3 shows the introduction of a fluid, such as a medicament, into the vessel from proximal sides of the balloon and expandable section, with the balloon and expandable section preventing the fluid from flowing proximally into the vessel;

FIG. 4 depicts the balloon and the expandable section of the embodiment of the system of FIG. 1 in a slightly less expanded state, which allows some of the fluid shown in FIG. 3 to flow proximally between the balloon and the wall of the vessel and may allow some of the fluid to flow proximally beyond the system;

FIG. 5 illustrates the embodiment of the system of FIG. 1 in a contracted state following the introduction of fluid into the vessel, with pressure having been removed from the balloon of the balloon catheter (i.e., the balloon has been deflate) and the expandable section of the expandable medical device has been allowed to contract to facilitate removal of the system from the vessel;

FIGS. 6-10 depict an embodiment of a system in which a length of an expandable section of an expandable medical device exceeds a length of a balloon of a balloon catheter and a position of the balloon within the expandable section may be changed, with FIG. 6 showing the balloon at a distal position within the expandable section, FIG. 7 showing the balloon at an intermediate position within the expandable section, FIG. 8 showing the balloon at a proximal position within the expandable section, FIG. 9 showing the balloon back at the intermediate position within the expandable section, and FIG. 10 showing the balloon back at the distal position within the expandable section.

### DETAILED DESCRIPTION

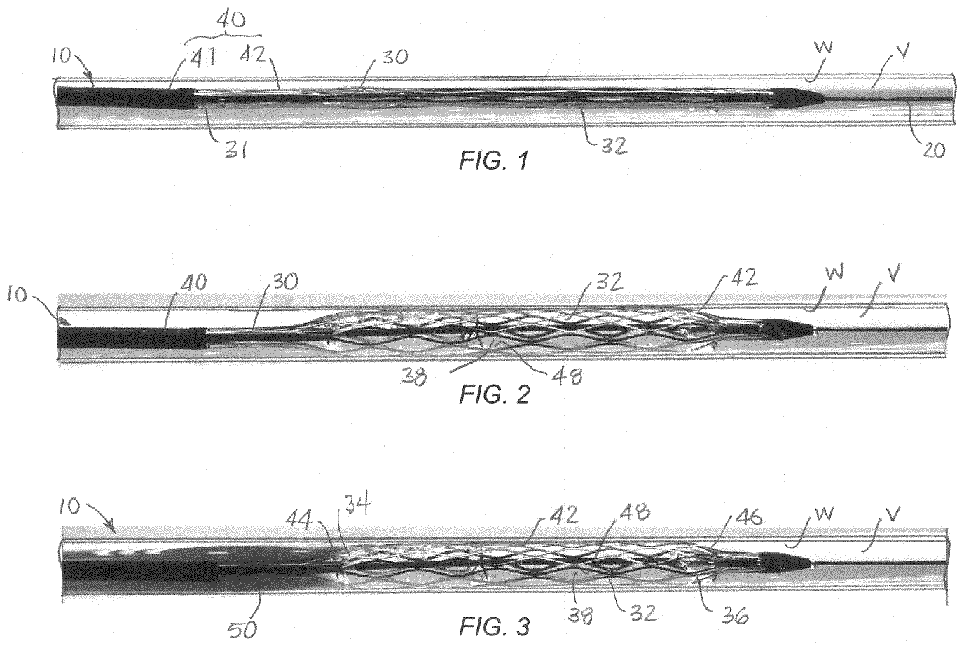

An embodiment of a system 10 according to this disclosure is shown in FIG. 1. The system 10 may include a balloon catheter 30 and an expandable medical device 40. In addition, the system 10 may include a guide wire 20, which may facilitate advancement of the balloon catheter 30 and/or the expandable medical device 40 into the body of a subject.

The balloon catheter 30 may comprise a percutaneous transluminal angioplasty (PTA) balloon catheter or the like. The balloon catheter 30 includes a catheter 31 and a balloon 32 at a distal location on the catheter 31. An interior of the balloon 32 may communicate with a lumen of the catheter 31 and receive a fluid (e.g., a gas or mixture of gases, a liquid etc.) from the lumen of the catheter 31. As fluid is introduced into the interior of the balloon 32, pressure within the interior of the balloon 32 increases, which expands or inflates the balloon 32. As fluid is removed from the interior of the balloon 32 (e.g., through the lumen of the catheter 31, etc.), pressure within the interior of the balloon 32 decreases, which contracts or deflates the balloon 32.

The expandable medical device 40 may comprise an exoskeleton device. Without limitation, the expandable medical device 40 may comprise an exoskeleton device of the type disclosed by U.S. patent application Ser. No. 16/174,205, filed Oct. 28, 2019 and titled EXOSKELETON DEVICE WITH EXPANDABLE SECTION FOR SCORING, U.S. patent application Ser. No. 16/540,046, filed on Aug. 13, 2019 and titled EXOSKELETON DEVICE WITH EXPANDABLE SECTION FOR SCORING, and U.S. patent application Ser. No. 16/579,770, filed Sep. 23, 2019 and titled EXPANDABLE EXOSKELETON DEVICES, the entire disclosures of which are hereby incorporated herein. Such an expandable medical device 40 is available from Transit Scientific, LLC, of Salt Lake City, Utah, U.S.A., as the XO SCORE sheath platform.

The expandable medical device 40 may and comprise a catheter 41 with an expandable section 42. The expandable section 42 may be located at or adjacent to a distal end of the catheter 41. A lumen of the catheter 41 of the expandable medical device 40 may receive the balloon catheter 30 while the balloon thereof is in a deflated or contracted state. When the balloon catheter 30 and the expandable medical device 40 are fully assembled, the catheter 31 of the balloon catheter 30 may reside within the catheter 41 of the expandable medical device 40 and the balloon 32 of the balloon catheter 30 may reside within the expandable section 42 of the expandable medical device 40. As the balloon 32 expands, the expandable section 42 may also expand (e.g., due to its construction and/or material, etc.). As the expandable section 42 contracts (e.g., from resilience from its construction and/or material, etc.), it may also collapse the balloon 32. The balloon 32 may be moveable within the expandable section 42. The balloon 32 may also be removable from the expandable section 42 and the balloon catheter 30 may be removable from the expandable medical device 40.

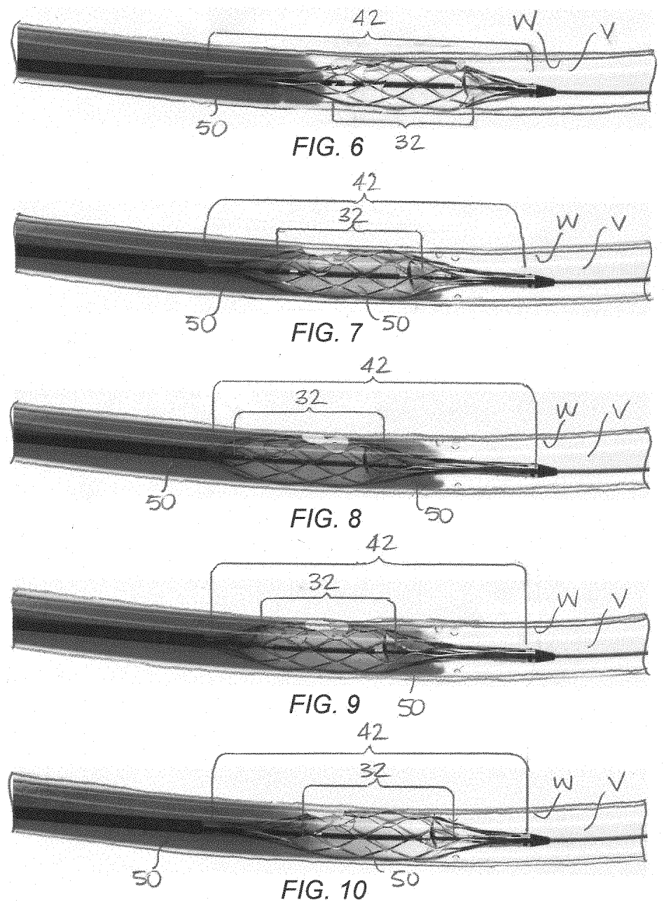

In some embodiments, such as that depicted by FIG. 1, the expandable section 42 of the expandable medical device 40 may have a length that slightly exceeds a length of the balloon 32 of the balloon catheter 30. As such, the balloon 32 may substantially extend along a length of an interior of the expandable section 42. In other embodiments, such as that depicted by FIG. 6-10, the balloon 32 may be significantly shorter than the expandable section 42, which may enable the balloon 32 to positioned at a plurality of different locations along the length of the expandable section 42. Without limitation, the balloon 32 may be about 90%, about 80%, about 75%, about 70%, about two-thirds, about 50%, about 40%, about one third, about 30%, about 25%, about 20% or about 10% as long as the expandable section, or within a range of lengths between any of the foregoing values, or within a range of lengths in which the foregoing values represent maximums (e.g., at most 90%, at most 80%, at most 75%, etc.).

FIG. 1 illustrates the system 10 and, more specifically, the balloon 32 and expandable section 42, at a desired location or a target location within a vessel or other space V within a subject's body. In FIG. 1, the balloon 32 and the expandable section 42 are in unexpanded states, or contracted states. While in their unexpanded states, at least part of the expandable section 42 is spaced apart from a wall W of the vessel or other space V.

In FIG. 2, the balloon 32 of the balloon catheter 30 and the expandable section 42 of the expandable medical device 42 of the system 10 are in expanded states. More specifically, as pressure is introduced into an interior of the balloon 32 (i.e., as the balloon 32 is inflated), outer surfaces of the balloon 32 force the expandable section 42 to expand outwardly. Expansion of the balloon 32 and the expandable section 42 reduces the spacing, or closing a gap, between the expandable section 42 and the wall W of the vessel or other space V. Pressure within the balloon 32 may be sufficient to cause the expandable section 42 to score the wall W. Pressure within the balloon 32 may be sufficient to cause portions of an outer surface 38 of the balloon 32 that are exposed through openings 48 in the expandable section 42 to substantially seal (i.e., allow for only tolerable amounts of leakage, etc.) or to seal against the wall W.

With portions of the outer surface 38 of the balloon 32 substantially sealed or sealed against the wall W of the vessel or other space V, the balloon 32 may substantially prevent (i.e., allow for only tolerable amounts of leakage, etc.) or prevent fluids from flowing past it. As depicted by FIG. 3, when the balloon 32 is inflated and portions of the outer surface 38 are sealed against the wall W of the vessel or other space V, a fluid 50, such as a drug or another substance, may be introduced into the vessel or other space V. The fluid 50 may be introduced into the vessel or other space V at or adjacent to proximal sides 34 and 44 of the balloon 32 and the expandable section 42, respectively, as shown in FIG. 3. Alternatively, the fluid 50 may be introduced into the vessel or other space V at or adjacent to distal sides 36 and 44 of the balloon 32 and the expandable section 42, respectively. In some embodiments, the fluid 50 may be introduced into the vessel or other space V through the catheter 41 of the expandable medical device 40 and exit the expandable medical device 40 at a location at the proximal side 44 of the expandable section 42, on the proximal side 34 of the balloon 32.

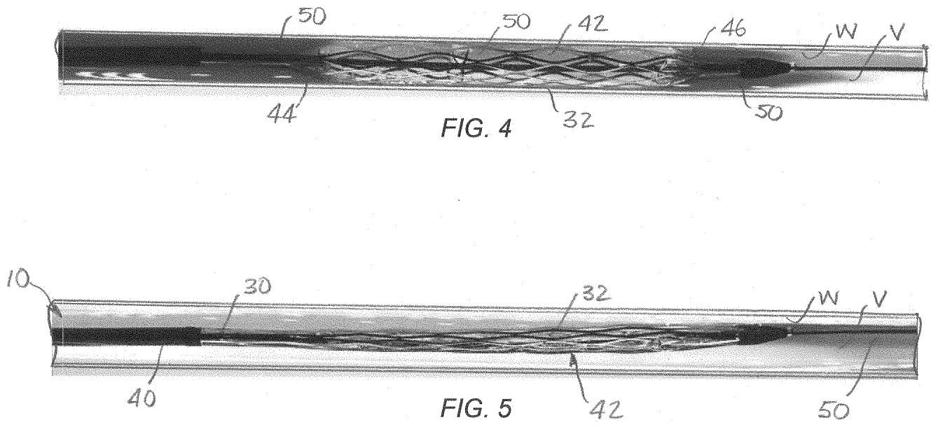

Referring now to FIG. 4, in some embodiments, as an alternative to causing portions of the outer surface 38 of the balloon 32 to seal against the wall W of the vessel or other space V to substantially prevent or prevent fluid (e.g., the fluid 50, etc.) from flowing past the balloon 32, the balloon 32 may be pressurized or inflated to an extent that allows for fluid to flow past the balloon 32, but provides control over the amount of the fluid 50 that can flow past the balloon 32 or the rate at which the fluid 50 or any other fluid can flow past the balloon 32. In other embodiments, once the fluid 50 has been introduced into the vessel or other space V, pressure within the balloon 32 may be reduced to allow the fluid 50 to flow beyond the balloon 32. In both of the preceding types of embodiments, portions of the outer surface 38 of the balloon 32 may contact the wall W of the vessel or other space V without sealing against the wall W or the outer surface 38 of the balloon 32 may be spaced apart from the wall W. The pressure within the interior of the balloon 32 may be controlled to limit the amount of fluid (e.g., the fluid 50, etc.) that may flow along and optionally beyond the balloon 32. As illustrated by FIG. 4, with reduced pressure in the balloon 32, the fluid (e.g., the fluid 50, etc.) is able to flow between the outer surface 38 of the balloon 32 and the wall W and may also flow onto an opposite side of the balloon 32 (e.g., the proximal sides 36 and 46 of the balloon 32 and expandable section 42, respectively, etc.).

In some embodiments, the balloon 32 may be slightly deflated and re-inflated once or repeatedly to enable fluid (e.g., the fluid 50, etc.) to flow between the expandable section 42 and the adjacent wall W of the vessel or other space V and to encourage the fluid to enter the wall W (e.g. by physically forcing the fluid into the wall W, into scores formed in the wall W by the expanded expandable section 42, by repeatedly scoring the wall W with the expandable section 42, etc.).

Once infusion is complete, the balloon 32 and expandable section 42 may be contracted, as shown in FIG. 5 and then withdrawn from the vessel or other space V and, thus, from the body of the subject.

Turning now to FIGS. 6-10, in embodiments where the length of the expandable section 42 of the expandable medical device 40 significantly exceeds a length of the balloon 32 of the balloon catheter 30, the balloon 32 may be moved to a plurality of discrete locations within the expandable section 42 to enable the balloon 32 to expand the expandable section 42 to a plurality of different expanded states, or configurations. Such movement may be back and forth. For example, such movement may occur as the balloon catheter 30 is pushed distally and pulled proximally relative to the expandable section 42 of the expandable medical device 40. For example, the balloon may be moved from a distal position within the expandable section 42 (see FIG. 6) to an intermediate position within the expandable section 42 (see FIG. 7), then to a proximal position within the expandable section 42 (see FIG. 8), then back to an intermediate position within the expandable section 42 (see FIG. 9), and then back to a distal position within the expandable section 42 (see FIG. 10). As another example, such movement may be from a proximal position within the expandable section 42 (see FIG. 8), to an intermediate position (see FIG. 9), to a distal position (see FIG. 10), back to the intermediate position (see FIG. 9), and back to the proximal position (see FIG. 8). As another alternative, such movement may be peristaltic in nature, involving a plurality of different intermediate positions as the balloon is moved distally and or proximally. In yet another alternative, such movement may simply alternate between a proximal position and a distal position. Movement of the balloon may include decreasing a pressure within (i.e., partially deflating) the balloon, changing a location of the balloon within the vessel or other space and/or within the expandable section of the expandable medical device, and increasing a pressure within (i.e., further inflating) the balloon. Movement of the balloon within the expandable section may force fluid (e.g. a drug, another substance, etc.) toward and/or into or otherwise encourage the fluid to flow into a wall of the vessel or other space within which the balloon and expandable section are located.

Although the foregoing description provides many specifics, these should not be construed as limiting the scopes of any of the appended claims, but merely as providing information pertinent to some specific embodiments that may fall within the scopes of the appended claims. Features from different embodiments may be employed in combination. In addition, the scopes of the appended claims may encompass other embodiments. All additions to, deletions from, and modifications of the disclosed subject matter that fall within the scopes of the claims are to be embraced by the claims.

What is claimed:

1. A method for introducing a fluid into a body of a subject, comprising:
introducing an expandable section of an expandable medical device to a desired location within an open space within the body of the subject;
introducing a balloon of a balloon catheter into the expandable section of the expandable medical device;
pressurizing the balloon to cause the balloon and the expandable section to expand and to contact a wall of the open space;
introducing a fluid into the open space on one side of the balloon, the balloon limiting flow of the fluid between the balloon and the wall and to an opposite side of the balloon.

2. The method of claim 1, wherein pressurizing the balloon causes the expandable section to expand in a manner that scores the wall of the open space.

3. The method of claim 1, wherein pressurizing the balloon comprises defining an amount of the fluid and/or a rate at which the fluid may flow between the balloon and the wall.

4. The method of claim 1, wherein pressurizing the balloon comprises causing portions of an outer surface of the balloon exposed through openings in the expandable section to seal against the wall.

5. The method of claim 4, wherein causing portions of the outer surface or the balloon exposed through the openings in the expandable section to seal against the wall comprises substantially preventing the fluid from flowing between the balloon and the wall.

6. The method of claim 1, further comprising:
reducing a pressure within the balloon to allow the fluid to flow between the balloon and the wall.

7. The method of claim 6, further comprising:
increasing a pressure within the balloon to cause the balloon to force the fluid into the wall and/or to score the wall to facilitate introduction of the fluid into the wall.

8. The method of claim 1, further comprising:
while maintaining a position of the expandable section within the open space, moving the balloon proximally and/or distally within the expandable section.

9. The method of claim 8, further comprising:
reducing a pressure within the balloon before moving the balloon.

10. The method of claim 9, further comprising:
after moving the balloon, re-inflating the balloon.

11. The method of claim 8, further comprising:
while maintaining a position of the expandable section within the open space, moving the balloon proximally and/or distally within the expandable section again.

12. A system for infusing a fluid into a body of a subject, comprising:
an expandable medical device with an expandable section;
a balloon catheter including a balloon with an outer surface that can seal against a wall of an open space within the body of the subject, the balloon having a length that enables it to be positioned at a plurality of discrete locations within the expandable section of the expandable medical device and to cause the expandable section to expand to a plurality of different configurations; and
the fluid.

13. The system of claim 12, wherein a length of the balloon is at most 75% of a length of the expandable section.

14. The system of claim 12, wherein a length of the balloon is at most 50% of a length of the expandable section.

15. The system of claim 12, wherein a length of the balloon is at most 25% of a length of the expandable section.

16. The system of claim 12, wherein the expandable medical device includes a catheter that carries the fluid to the expandable section, from which the fluid may exit the expandable medical device an enter the open space.

17. The system of claim 12, further comprising:
a pressurization component that controls a pressure within the balloon.

18. The system of claim 12, wherein the expandable section resiliently contracts.

19. The system of claim 12, wherein resilient contraction of the expandable section causes the balloon to collapse.

20. The system of claim 12, wherein the expandable section, when expanded within the open space, can score the wall that defines the open space.
